(19) United States
(12) Patent Application Publication
(10) Pub. No.: US 2012/0245667 A1
(43) Pub. Date: Sep. 27, 2012

(54) ELECTRODE PAD AND CONNECTORS FOR ELECTROTHERAPY DEVICES

(75) Inventors: Miles Alexander Jordan, London (GB); Sarah Louise Beeby, London (GB); Richard Adam Nagle, London (GB)

(73) Assignee: WOUND SOLUTIONS LTD., London (GB)

(21) Appl. No.: 13/392,746

(22) PCT Filed: Aug. 27, 2010
(86) PCT No.: PCT/IB10/02374

§ 371 Date: Jun. 14, 2012

Related U.S. Application Data
(60) Provisional application No. 61/237,541, filed on Aug. 27, 2009.

(51) Int. Cl. A61N 1/04; A61F 13/00

(52) U.S. Cl. 607/149; 607/115

(57) ABSTRACT

An electrode pad for an electrotherapy device is configured to be placed outside of a wound area for a patient. The electrode pad can include a first lobe of material and a second lobe of the material connected together at a non-conductive attachment area. The electrode pad can have a first electrode located within the first lobe of material, a second electrode located within the second lobe of material, a first wire that is electrically connected to the first electrode, and a second wire that is electrically connected to the second electrode. The first and second lobes are configured to be movable with respect to each other about the attachment area, and the first and second wires are configured to form an electrical connection between the first and second electrodes and the electrotherapy device. A ribbon cable can be configured to be attached to the attachment area at any angle.

### CROSS-REFERENCE TO RELATED APPLICATIONS

This application claims the benefit of priority to U.S. Provisional Application No. 61/237,541, filed on Aug. 27, 2009, titled “Ambulatory Electrotherapy Device Connectors,” the disclosure of which is incorporated by reference in its entirety.

### TECHNICAL FIELD

This disclosure generally relates to electrode pads and connectors for electrotherapy devices.

### BACKGROUND

Electrotherapy devices can be for the treatment of wounds, such as for the treatment of venous leg ulcers. Electrotherapy devices can deliver current to the wound area, which can provide for the stimulation of non-healing wounds, as well as for providing analgesic effects.

### SUMMARY

This specification generally relates to electrode pads and connectors for electrotherapy devices.

In general, an aspect of the subject matter described in this specification can be embodied an electrode pad for an electrotherapy device. The electrode pad has a first lobe of material and a second lobe of the material connected together at an attachment area. The attachment area is electrically conductive. The electrode pad has a first electrode located within the first lobe of material, a second electrode located within the second lobe of material, and two or more wires from an electrotherapy device coupled to the attachment area. The first and second electrodes are electrically connected to the two or more wires through the attachment area. The electrode pad is configured to be positioned outside of a wound area on a patient to run a current through the wound area.

These and other embodiments can each optionally include one or more of the following features. The two or more wires can be formed within a ribbon cable. The ribbon cable can be made of polyester and Mylar. The two or more wires in the ribbon cable can be configured to be in printed conductive tracks that extend in a direction that is substantially parallel to a longitudinal axis of the ribbon cable. The attachment area of the electrode pad can include first and second printed conductors corresponding to the first and second electrodes. The electrode pad can be configured to be fixably attached to the ribbon cable at any angle. For an axis of the electrode pad that intersects the first and second lobes and the longitudinal axis of the ribbon cable, the ribbon cable can be configured to be attached to the attachment area of the electrode pad at any angle between the axis of the electrode pad that intersects the first and second lobes and the longitudinal axis of the ribbon cable. The ribbon cable can be configured to rotate around the attachment area of the electrode pad in a circular path. The first and second electrodes can include printed conductors. The electrode pad can be configured to be affixed to a body part of a patient by compression dressing wrapped over at least a part of the electrode pad. The first and second lobes of material can include a non-conductive material. The lobes can include foam. The first and second electrodes can be configured to be in contact with a layer of conductive gel. The electrode pad can be configured to be placed at an area on the patient that is beside the wound area to cover up to about one half of an area outside of an edge of the wound area, for which the electrode pad is configured to leave about another half of the area outside of the edge of the wound area uncovered for a placement of a second electrode pad.

In general, another aspect of the subject matter described in this specification can be embodied an electrode pad for an electrotherapy device, where the electrode pad is configured to be placed outside of a wound area for a patient. The electrode pad includes a first lobe of material and a second lobe of the material connected together at an attachment area. The electrode pad includes a first electrode located at the first lobe of material, for which the first electrode includes a first printed conductor in contact with the first lobe of material, and a second electrode located at the second lobe of material, for which the second electrode comprises a second printed conductor in contact with the second lobe of material. The electrode pad includes a first metal pin that is electrically connected to the first electrode, for which the first pin is configured to extend outwards from the electrode pad in a direction that is away from the wound area. The electrode pad includes a second metal pin that is electrically connected to the second electrode, for which the second pin is configured to extend outwards from the electrode pad in a direction that is away from the wound area. The first pin is configured to be attached to a first connector, and the second pin is configured to be attached to a second connector, for which the first and second connectors are configured to form an electrical connection between the first and second electrodes and the electrotherapy device.

These and other embodiments can each optionally include one or more of the following features. The first and second metal pins can be configured to be fastened to either the first or second connectors. The first and second metal pins can be configured to be fixably attached to either the first or second connectors. The first and second pins can have a length of about 3 mm. The first and second pins each can have a tip that has a rounded point. The first and second pins can be configured to extend through a layer of compression dressing wrapped around the electrode pad. The first and second pins can be configured to connect to the first and second connectors with at least part of the layer of the compression dressing positioned between the electrode pad and the first and second connectors. The first and second connectors can be configured to have a button-like connection to clip onto to either the first or second metal pins. The first and second lobes of material can be non-conductive. The attachment area can be less than an area of either of the first or second lobes of material. The attachment area can be located between the first and second lobes. The first or second lobes of material can be non-conductive.

In general, another aspect of the subject matter described in this specification is embodied an electrode pad for an electrotherapy device, for which the electrode pad is configured to be placed outside of a wound area for a patient. The electrode pad includes a first lobe of material and a second lobe of the material connected together at a non-conductive attachment area, a first electrode located within the first lobe of material, a second electrode located within the second lobe of material, a first wire that is electrically connected to the first electrode, and a second wire that is electrically connected to the second electrode. The first and second lobes are configured to be movable with respect to each other about the attachment area. The first and second wires are configured to form an electrical connection between the first and second electrodes and the electrotherapy device.

These and other embodiments can each optionally include one or more of the following features. The non-conductive attachment area can include a strip of foam material, for which a first end of the strip is connected to the first lobe and a second end of the strip is connected to the second lobe. A size of the strip can be less than a size of either of the first or second lobes of material, for which a maximum height of the strip between the first and second lobes can be about 5 mm to 6 mm. The first and second wires can be configured to enter an exterior side of the first and second lobes of material, for which the exterior side of the first and second lobes of material can be in a direction that faces away from the wound area of the patient. An interior side of the first and second lobes of material is in a direction that faces the wound area of the patient, for which the first and second lobes of material can be configured to be in contact with the first and second electrodes. The first and second electrodes can be configured to receive a layer of conductive gel. The first and second lobes can be configured to be movable with respect to each other about the attachment area to have one side of the electrode pad to form a substantially straight line. The first and second lobes can be configured to be movable with respect to each other about the attachment area to have one side of the electrode pad to form a substantially curved line. The first and second lobes can be configured to be movable with respect to each other about the attachment area to have one side of the electrode pad to form a V-shaped line. The first and second lobes can be configured to be movable with respect to each other about the attachment area such that the first and second lobes are configured to pivot around the attachment area. The electrode pad can be configured to be attached to the patient by having a layer of compression dressing wrapped around the electrode pad.

The details of one or more embodiments of the subject matter described in this specification are set forth in the accompanying drawings and the description below. Other features and aspects of the subject matter will become apparent from the description, the drawings, and the claims.

### BRIEF DESCRIPTION OF THE DRAWINGS

FIG. 1 depicts a diagram of an implementation of an electrotherapy device mounted on a leg of a patient.

FIG. 2A depicts an example of the electrode pad with a ribbon cable.

FIG. 2B depicts an example of an implementation that features a connection made by a printed ribbon cable attached to the pad using a z-axis conductive adhesive.

FIG. 2C depicts an example of the implementation of FIG. 2B that shows the arrangement of the ribbon cable.

FIG. 2D depicts an example of two electrode pads, each attached to a ribbon cable, positioned underneath a compression dressing on a leg of a patient.

FIGS. 3A and 3B depict diagrams with views of an implementation of that includes conductive lobes with electrode pads.

FIG. 3C depicts a diagram for an application of a compression dressing and the electrode pads to the leg.

FIG. 4A depicts a diagram of an implementation of an electrode pad.

FIGS. 4B and 4C depict diagrams of the electrode pad of FIG. 4A positioned around wound areas.

Like reference numbers and designations in the various drawings indicate like elements.

### DETAILED DESCRIPTION

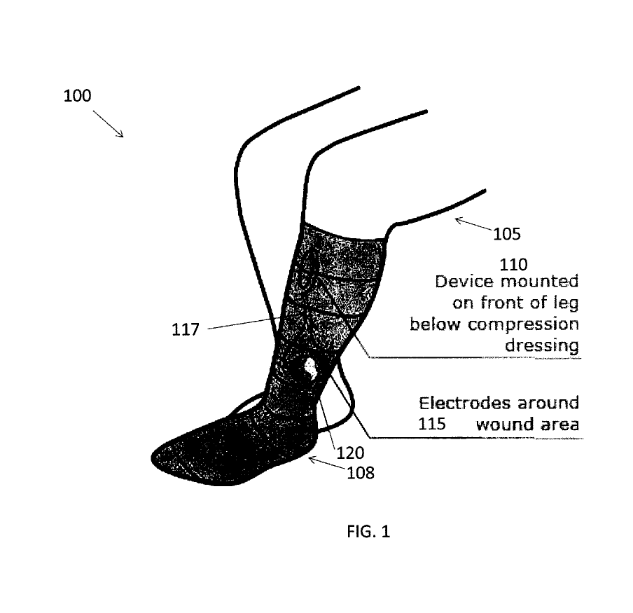

FIG. 1 depicts a diagram 100 of an implementation of an electrotherapy device 110 mounted on the leg 105 of a person. The electrotherapy device 110 is affixed to a patient by being positioned under a compression dressing 108. The electrotherapy device 110 is used with electrode pads, which includes two or more electrodes. The electrotherapy device 110 is connected to electrodes 1150 positioned around the wound area 120. The electrode pads can be placed surrounding, but not touching, the wound area 120. As described in one or more figures below, each electrode can have lead wires terminating in a connector. The wires can be plugged into the electronic module at the start of the therapy. The electrotherapy device 110 can be used in conjunction with compression therapy for the treatment of wounds, such as for the treatment of venous leg ulcers.

The electrotherapy device 110 can be any type of device that delivers signals to the body for healing. For example, the electrotherapy device 110 can be an ambulatory electrotherapy device that can have levels of feedback that enables patients, clinicians, and/or practitioners to access device information. The electrotherapy device information can include electrotherapy data, and data regarding the patient's adherence to a recommended mobility program to enhance the healing process via movements and leg elevation. The electrotherapy device 110 can use electrotherapy, in combination with elevation and/or motion/activity monitoring, analysis, and feedback to enhance the healing of the wound area. The electrotherapy device 110 can have a pressure/compression sensor, vibration alerts, and activity analysis feedback software. In FIG. 1, the electrotherapy device 110 includes the electronics for the electrotherapy, monitoring and compliance features, and is connected to the electrode pads underneath the compression dressing 108. While a leg is presented in the drawings, the implementations in this disclosure may be applied to other parts of the body (e.g., arm, hand, etc.).

In some embodiments, the electrotherapy device can have an electric module that is built into a case that fits onto a patient's leg at an area in compression dressing, in which the device can be fitted below and to the outside of a compression dressing. In one embodiment, the electrotherapy device can fit on top of a number of layers of compression dressing, positioned at the top end of the tibia below the knee, and held in place by the bandage's outer layer. The compression dressing can include various types of compression dressing.

The electronics within the electrotherapy device can include a number of components. For example, the electronics can include an electrotherapy generator, a motion sensor, control and display electronics, and a battery for power. In some embodiments, sensors can be used for the feedback mechanism of the device. Results from the sensors can be processed and used to provide motivation to the patient, to control the electrotherapy process, and to inform the practitioner with data for the therapy. In some embodiments, light indicators (e.g., light emitting diodes, LEDs) can be on the device to provide feedback and alerts to the patient. The indicators can provide motivation to the patient to keep moving, based on input from the motion sensor. Discrete patient feedback can also be provided by a vibration alarm, reminding the patient of the need to take an action. In some embodiments, the electrotherapy generator may be further equipped to sense unduly high resistance between electrodes, enabling the patient to be alerted to urgent issues with the treatment. In some embodiments, one or more switches can be used to obtain information regarding the operation of the device over a period of time, as well as the movements of the patient. Some or all of the switches can be practitioner-operated switches. In some embodiments, one or more switches can be used to start operation of the device and to detect whether the unit is properly incorporated in the bandage under compression.

In some embodiments, the current used in the wires that attach to the electrode pads can include a DC current, an AC current, a pulsed current, or a combination of various current types. For example, micro-currents with specially-tailored waveforms can be generated across the electrodes using the electrotherapy generator. Various current and waveform schemes are possible. In some embodiments, each treatment for a patient may involve several phases. In one example, each phase may have a unipolar square wave current that may be commutated every few seconds to avoid the presence of an average direct-current component. Each treatment may last a predetermined amount of minutes, and may be in a predetermined number of phases. The treatments can have various phases, frequencies, and current amplitudes. In some embodiments, treatments may be carried out once in a predetermined number of hours, where each treatment may use an alternate pair of pads from the pair used in a preceding treatment. Other embodiments may have other types of treatments, each involving different phases, currents, and frequencies, among other factors.

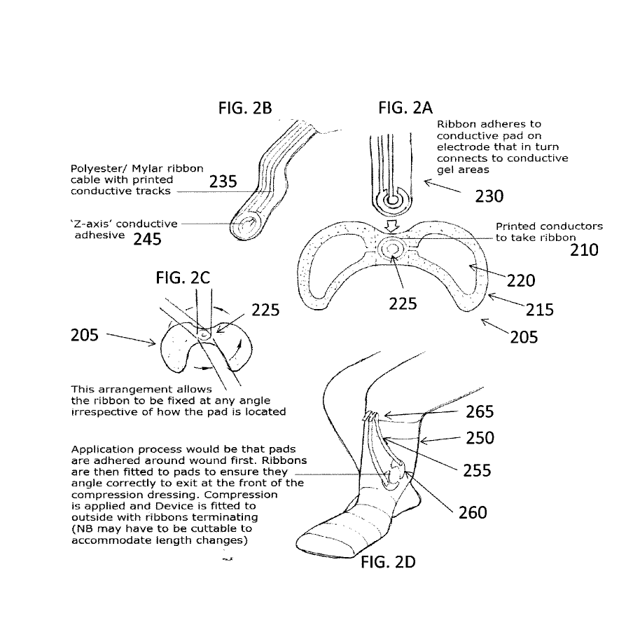

Some embodiments of an electrotherapy system can use ribbon cables instead of wires. FIG. 2A depicts an example of the electrode pad 205 with a ribbon cable 230. The ribbon cable 230 is adhered to the electrode pad 205 and is enclosed up inside of the compression dressing. The electrode pad 205 has printed conductors 210 to take the ribbon. Some embodiments can use thinner, more flexible (and possibly flatter) wires that can remain under the dressing without concern for contusions. In some embodiments, there can be sheathing wires in addition to padding, in which the sheathing wires can be positioned under the compression dressing.

The electrode pad 205 has an attachment area 225 for the wire corrections to the electrotherapy device. The pad includes two lobes, with an electrode 220 placed within each lobe, for which each lobe has an electrical connection to the electrotherapy device via the ribbon cable 230. Each lobe can also include foam 215 and a conductive gel, where the conductive gel may touch the skin of the leg and/or be located on an interior section of each lobe. The position of the foam 215, conductive gel, and the electrode 220 in each lobe can be stacked and arranged in a number of configurations. For example, the electrode pad may have a conductive gel at the skin of the patient, a layer of foam positioned behind the conductive gel, and the electrode positioned behind the layer of foam. In another example, the electrode pad may have a conductive gel at the skin, the electrode positioned behind the conductive gel, and a layer of foam positioned behind the electrode. The electrode pad may be constructed of other material, and may have other (or additional) layers of material as well.

FIG. 2B depicts an example of an implementation that features a connection made by a printed ribbon cable attached to the attachment area 225 of the electrode pad using a z-axis conductive adhesive 245 (e.g., a conductive adhesive located at an axis that, from one viewpoint, comes out of (or goes into) the page along a z-axis). The printed ribbon cable can be a polyester/Mylar ribbon cable 235 with printed conductive tracks. FIG. 2C depicts an example of the implementation of FIG. 2B that shows the arrangement of the ribbon cable 205 when the z-axis conductive adhesive 245 is connected to the ribbon cable 205. The connection at the attachment area 225 with the ribbon cable 235 is circular, and therefore, can be made at any angle to accommodate electrode placement on any position on the leg, irrespective of how the electrode pad is located on the leg.

FIG. 2D depicts an example of two electrode pads 260, each attached to a ribbon cable 255, positioned underneath the compression dressing 250 on the leg of a patient. The ribbon cables 255 extend to the outside 265 of the compression dressing 250. When applying the electrode pads 260 to the leg, the ribbon cables 255 are attached to the pads, and the electrode pads 260 are placed around the wound area. The ribbon cables 255 are attached to the electrode pads 260 such that they are at an angle to exit at the top and/or front of the compression dressing 250. The compression dressing 250 is applied to the leg, and the electrotherapy device is fitted to the outside of the compression dressing 250 with the ribbon cables terminating at the outside 265 of the compression dressing 250. The ribbon cables 265 may be cut to fit an appropriate length of the leg, length of the compression dressing around the leg, and/or the length of connection to the electrotherapy device.

In some implementations of FIGS. 2A-2D, the electrode pad for the electrotherapy device can be described as having a first lobe of material and a second lobe of the material connected together at an attachment area, for which the attachment area is electrically conductive. The first and second electrodes are located within the first and second lobes of material, respectively. Two or more wires from an electrotherapy device are coupled to the attachment area, for which the first and second electrodes are electrically connected to the two or more wires through the attachment area. The electrode pad is configured to be positioned outside of a wound area on a patient to run a current through the wound area.

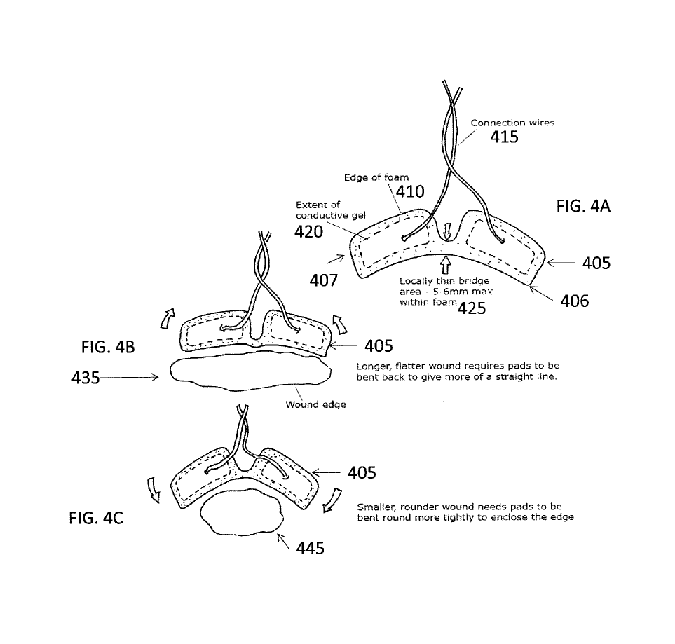

In some implementations of FIGS. 2A-2D, there are two or more wires formed within the ribbon cable (e.g., a ribbon cable formed of both polyester and Mylar). Two or more of the wires in the ribbon cable can be printed in conductive tracks that extend in a direction that is substantially parallel to a longitudinal axis of the ribbon cable. The attachment area of the electrode pad can include first and second printed conductors corresponding to the first and second electrodes. The electrode pad can be configured to be fixably attached to the ribbon cable at any angle. For an axis of the electrode pad that intersects the first and second lobes and the longitudinal axis of the ribbon cable, the ribbon cable can be configured to be attached to the attachment area of the electrode pad at any angle between the axis of the electrode pad that intersects the first and second lobes and the longitudinal axis of the ribbon cable. For example, the ribbon cable can be configured to rotate around the attachment area of the electrode pad in a circular path. Also, the first and second electrodes can include printed conductors. As shown in FIG. 2D, for example, the electrode pad can be configured to be affixed to a body part of a patient by compression dressing wrapped over at least a part of the electrode pad. The first and second lobes of material can include a non-conductive material in some implementations. The lobes can include foam, and the first and second electrodes can be configured to be in contact with a layer of conductive gel. The layer of conductive gel can be in contact with the skin of the patient to assist in providing an electrical connection to the skin. As illustrated with respect to FIGS. 4B and 4C, for example, the electrode pad can be configured to be placed at an area on the patient that is beside the wound area to cover up to about one half of an area outside of an edge of the wound area. The electrode pad can be configured to leave about another half of the area outside of the edge of the wound area uncovered for a placement of a second electrode pad. The two electrode pads can surround the wound area, and current(s) can flow through the wound area and between the electrodes of the oppositely-placed electrode pads.

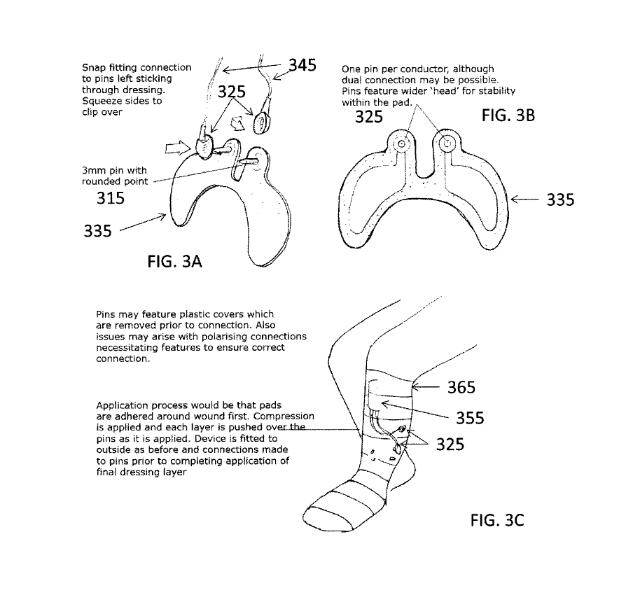

FIGS. 3A and 3B depict views of an implementation of that includes connectors 325 and two conductive lobes in the electrode pad 335. FIG. 3A shows a profile view of the electrode pad 335, and FIG. 3B shows a front view of the electrode pad 335. Each of the conductive lobes has a pin 315 that is configured to couple to a connector 325 to connect a wire or a conductive track in the ribbon cable 345 between the electrotherapy device and each one of the conductive lobes. To form a connection to the conductive lobes, the pins 315 can be configured to have a snap fitting connection with the connector 325. In some implementations, the snap fitting connection may be a button-like fitting (e.g., like the button-like fitting that clips or snaps the connection parts together).

Some embodiments include metal pins that pass through the compression dressing as it is applied to the patient, for which the connections can be made by clipping the connectors onto the ends of the pins. Some embodiments may have an electrode pad made with a stainless steel/aluminum polished pin to penetrate layers of compression dressing as they are applied to the wound area. Some other embodiments may use other types of materials and/or metals for the pin. The electrode pad also can allow wires or the ribbon cable to be reused, in particular if the wires or the ribbon cable is connected to a rubber jacket enclosing the device. In some embodiments, the pins 315 may be about 3 mm in length with a rounded point, for example. Some embodiments may have two or more pins per conductive lobe. The pins may have a plastic covers that can be removed prior to connecting them to the electrode pad. The connectors may connect to either pin so that, for embodiments where the polarity of the pen is a design feature, the connectors can be easily switched on the pins to have the correct polarity.

As shown in FIG. 3C, the application of the electrode pads to the leg may be similar as described above with respect to FIG. 2D. The electrode pads are first applied to the skin around the wound area, then the compression dressing 365 is applied, for which the layer(s) of the compression dressing may be placed over the pins as it is applied to the leg. Then, the electrotherapy device 355 can be fitted to the outside of the compression dressing 365 (or within at least a layer of compression dressing 365), and connectors can be connected to the pins before a final compression dressing layer is applied to cover the electrotherapy pads, the electrotherapy device 355, and the associated wires or ribbon cables.

Some implementations of FIGS. 3A-3C may be described as an electrode pad for an electrotherapy device that is configured to be placed outside of the wound area for the patient. The electrode pad can be described as having a first lobe of material and a second lobe of the material connected together at an attachment area, and a first electrode located at the first lobe of material, as well as a second electrode located at the second lobe of material. The first electrode has a first printed conductor in contact with the first lobe of material, and the second electrode has a second printed conductor in contact with the second lobe of material. The electrode pad can be described as having a first metal pin that is electrically connected to the first electrode, and a second metal pin that is electrically connected to the second electrode, for which the first pin and second pins are configured to extend outwards from the electrode pad in a direction that is away from the wound area (e.g., a direction substantially parallel to a z-axis). The first pin is configured to be attached to a first connector, and the second pin is configured to be attached to a second connector, for which the first and second connectors are configured to form an electrical connection between the first and second electrodes and the electrotherapy device.

Some implementations of FIGS. 3A-3C may be described as having at least some of the following features. The first and second metal pins can be configured to be connected (e.g., fastened, clipped, fixably attached) to either the first or second connectors. The first and second pins can have a tip that has a rounded point, and the first and second pins can be about 3 mm long, for which the length may range from about 0.5 mm to 6 mm, for example. In some implementations, the pins can be long enough such that the first and second pins can be configured to extend through a layer of compression dressing wrapped around the electrode pad, and the first and second pins can connect to the first and second connectors such that at least part of the compression dressing is positioned between (e.g., sandwiched between) the electrode pad and the first and second connectors. The first and second lobes of material may be non-conductive in some implementations. The attachment area is generally less than an area of either of the first or second lobes of material, for which the attachment area is located between the first and second lobes.

FIG. 4A depicts a diagram of an electrode pad 405 that is configured so that various electrodes can be positioned in various positions around the wound area, and can be used to pass various types of current in various ways across the wound area. The electrode pad 405 features a flexible pad that has two lobes 406, 407 physically connected by a thin bridge area or strip 425 of material. An electrode can be placed within each lobe, in which each lobe has an electrical connection wire 415 from the electrotherapy device extending into it. The electrode pad 405 includes foam and an electrically conductive gel, where an area of the conductive gel 420 on the pad is less than the edge of the foam 410. The dual-lobe electrode pad can have the ability to be shaped around the wound area to conform to the shape of the wound area.

FIGS. 4B and 4C depict diagrams of the electrode pad of FIG. 4A positioned around wound areas. As illustrated above, a strip 425 of foam material is left as a joint between the conductive pads. The strip 425 allows the dual-lobe electrode pad to be bent to conform to the edge of the wound area. If the wound area 435 is substantially flat, as in the wound area 435 of FIG. 4B, then the dual-lobe electrode pad can be positioned close to the wound area to form a straight line at the edge of the wound area. If the wound area 445 is more rounded in shape, as in the wound area 445 of FIG. 4C, then the dual-lobe electrode pad can be bent at the strip to form a more rounded, curved, or contoured shape at the edge of the wound area 445. Another dual-lobe electrode pad can be placed on an opposite side of the wound area 445 to allow currents to flow through the wound area and between the two oppositely-placed dual-lobe electrode pads.

The flexible pad can fit closely to the wound, regardless of the shape of the wound. This embodiment also can permit clinicians to position the flexible pad in various locations around the wound area instead of the same location. This may also alleviate some concerns of clinicians with respect to skin fragility because the electrodes do not have to be repeatedly placed in the same location on the patient.

As illustrated above, the strip can be formed as a joint between the conductive pads at a bottom portion of the conductive pads. In other embodiments, the strip can be formed as a joint between the conductive pads at a top or center/middle portion of the conductive pads. In some embodiments, the strip can be formed of other materials, including any suitable material to allow the conductive pads to remain physically connected.

In some implementations, for example, two dual-lobe electrode pads can be placed around the wound area, where various current schemes can be employed to have a first current from one electrode in one dual-lobe pad to flow through the wound area into at least one electrode in another oppositely-placed pad, and the oppositely-placed dual-lobe pad can have at least one electrode to have a second current to flow from the oppositely-placed dual-lobe pad, through the wound area and into one of the electrodes of the dual-lobe pad. For instance, currents can travel in one or more directions across the wound area and between various electrodes in oppositely-placed electrode pads around the wound area.

Some implementations of FIGS. 4A-4C may be described as an electrode pad for an electrotherapy device that is configured to be placed outside of a wound area for a patient. In these implementations, the electrode pad can be described as having a first lobe of material and a second lobe of the material connected together at a non-conductive attachment area, for which there is a first electrode located within the first lobe of material, a second electrode located within the second lobe of material, a first wire that is electrically connected to the first electrode; and a second wire that is electrically connected to the second electrode. In these implementations, the first and second lobes are configured to be movable with respect to each other about the attachment area, and the first and second wires are configured to form an electrical connection between the first and second electrodes and the electrotherapy device.

Some implementations of FIGS. 4A-4C may be described as having one or more of the following features. The non-conductive attachment area can be a strip of foam material, for which a first end of the strip is connected to the first lobe and a second end of the strip is connected to the second lobe. The size of the strip can be generally less than a size of either of the first or second lobes of material, for which a maximum height of the strip between the first and second lobes is about 5 mm to 6 mm in some implementations. The first and second wires can be configured to enter an exterior side of the first and second lobes of material, for which the exterior side of the first and second lobes of material is in a direction that faces away from the wound area of the patient. An interior side of the first and second lobes of material is generally in a direction that faces the wound area of the patient. The first and second lobes of material can be configured to be in contact with the first and second electrodes, and the first and second electrodes can be configured to receive the layer of conductive gel. In these implementations, as shown in the examples of FIGS. 4A-4C, the first and second lobes are configured to be movable with respect to each other about the attachment area to have one side of the electrode pad to form a substantially straight line, a substantially curved line, or even a V-shaped line (or a U-shaped, L-shaped, or C-shaped line). In these implementations, the first and second lobes can be configured to pivot around the attachment area. As with the other above-described electrode pads, the electrode pad of FIGS. 4A-4C is configured to be attached to the patient by having a layer of compression dressing wrapped around the electrode pad.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. The connectors, wires, electrode pads, and ribbon cables of FIGS. 2A-4C can be used with implementations and/or devices other than the electrotherapy device described above.

What is claimed is:

1. An electrode pad for an electrotherapy device, the electrode pad comprising:
a first lobe of material and a second lobe of the material connected together at an attachment area, wherein the attachment area is electrically conductive;
a first electrode located within the first lobe of material;
a second electrode located within the second lobe of material; and
two or more wires from an electrotherapy device coupled to the attachment area, wherein the first and second electrodes are electrically connected to the two or more wires through the attachment area, wherein the electrode pad is configured to be positioned outside of a wound area on a patient to run a current through the wound area.

2. The electrode pad of claim 1, wherein the two or more wires are formed within a ribbon cable.

3. The electrode pad of claim 2, wherein the ribbon cable comprises polyester and Mylar, wherein the two or more wires in the ribbon cable further are configured to be in printed conductive tracks that extend in a direction that is substantially parallel to a longitudinal axis of the ribbon cable.

4. The electrode pad of claim 3, wherein the attachment area of the electrode pad comprises first and second printed conductors corresponding to the first and second electrodes, and wherein the electrode pad is configured to be fixably attached to the ribbon cable at any angle.

5. The electrode pad of claim 3, wherein for an axis of the electrode pad that intersects the first and second lobes and the longitudinal axis of the ribbon cable, the ribbon cable is configured to be attached to the attachment area of the electrode pad at any angle between the axis of the electrode pad that intersects the first and second lobes and the longitudinal axis of the ribbon cable.

6. The electrode pad of claim 5, wherein the ribbon cable is configured to rotate around the attachment area of the electrode pad in a circular path.

7. The electrode pad of claim 1, wherein the first and second electrodes comprises printed conductors.

8. The electrode pad of claim 1, wherein the electrode pad is configured to be affixed to a body part of a patient by compression dressing wrapped over at least a part of the electrode pad, wherein the first and second lobes of material comprises a non-conductive material.

9. The electrode pad of claim 1, wherein the lobes comprise foam, and wherein the first and second electrodes are configured to be in contact with a layer of conductive gel.

10. The electrode pad of claim 1, wherein the electrode pad is configured to be placed at an area on the patient that is beside the wound area to cover up to about one half of an area outside of an edge of the wound area, wherein the electrode pad is configured to leave about another half of the area outside of the edge of the wound area uncovered for a placement of a second electrode pad.

11. An electrode pad for an electrotherapy device, the electrode pad being configured to be placed outside of a wound area for a patient, the electrode pad comprising:
a first lobe of material and a second lobe of the material connected together at an attachment area;
a first electrode located at the first lobe of material, wherein the first electrode comprises a first printed conductor in contact with the first lobe of material;
a second electrode located at the second lobe of material, wherein the second electrode comprises a second printed conductor in contact with the second lobe of material;
a first metal pin that is electrically connected to the first electrode, wherein the first pin is configured to extend outwards from the electrode pad in a direction that is away from the wound area; and
a second metal pin that is electrically connected to the second electrode, wherein the second pin is configured to extend outwards from the electrode pad in a direction that is away from the wound area,
wherein the first pin is configured to be attached to a first connector, and the second pin is configured to be attached to a second connector, and wherein the first and second connectors are configured to form an electrical connection between the first and second electrodes and the electrotherapy device.

12. The electrode pad of claim 11, wherein the first and second metal pins are configured to be fastened to either the first or second connectors.

13. The electrode pad of claim 11, wherein the first and second metal pins are configured to be fixably attached to either the first or second connectors.

14. The electrode pad of claim 11, wherein the first and second pins comprise a length of about 3 mm.

15. The electrode pad of claim 11, wherein the first and second pins each comprises a tip that has a rounded point.

16. The electrode pad of claim 11, wherein the first and second pins are configured to extend through a layer of compression dressing wrapped around the electrode pad, and wherein the first and second pins are configured to connect to the first and second connectors with at least part of the layer of the compression dressing positioned between the electrode pad and the first and second connectors.

17. The method of claim 11, wherein the first and second connectors are configured to have a button-like connection to clip onto to either the first or second metal pins.

18. The method of claim 11, wherein the first and second lobes of material are non-conductive, and wherein the attachment area is less than an area of either of the first or second lobes of material, and wherein the attachment area is located between the first and second lobes, wherein the first or second lobes of material is non-conductive.

19. An electrode pad for an electrotherapy device, the electrode pad being configured to be placed outside of a wound area for a patient, the electrode pad comprising:
a first lobe of material and a second lobe of the material connected together at a non-conductive attachment area;
a first electrode located within the first lobe of material;
a second electrode located within the second lobe of material;
a first wire that is electrically connected to the first electrode; and
a second wire that is electrically connected to the second electrode,
wherein the first and second lobes are configured to be movable with respect to each other about the attachment area, and
wherein the first and second wires are configured to form an electrical connection between the first and second electrodes and the electrotherapy device.

20. The electrode pad of claim 19, wherein the non-conductive attachment area comprises a strip of foam material, wherein a first end of the strip is connected to the first lobe and a second end of the strip is connected to the second lobe.

21. The electrode pad of claim 20, wherein a size of the strip is less than a size of either of the first or second lobes of material, wherein a maximum height of the strip between the first and second lobes is about 5 mm to 6 mm.

22. The electrode pad of claim 20, wherein the first and second wires are configured to enter an exterior side of the first and second lobes of material, wherein the exterior side of the first and second lobes of material is in a direction that faces away from the wound area of the patient.

23. The electrode pad of claim 22, wherein an interior side of the first and second lobes of material is in a direction that faces the wound area of the patient, wherein the first and second lobes of material are configured to be in contact with the first and second electrodes, wherein the first and second electrodes are configured to receive a layer of conductive gel.

24. The electrode pad of claim 19, wherein the first and second lobes are configured to be movable with respect to each other about the attachment area to have one side of the electrode pad to form a substantially straight line.

25. The electrode pad of claim 19, wherein the first and second lobes are configured to be movable with respect to each other about the attachment area to have one side of the electrode pad to form a substantially curved line.

26. The electrode pad of claim 19, wherein the first and second lobes are configured to be movable with respect to each other about the attachment area to have one side of the electrode pad to form a V-shaped line.

27. The electrode pad of claim 19, wherein the first and second lobes are configured to be movable with respect to each other about the attachment area such that the first and second lobes are configured to pivot around the attachment area.

28. The electrode pad of claim 19, wherein the electrode pad is configured to be attached to the patient by having a layer of compression dressing wrapped around the electrode pad.
